(19) United States
(12) Patent Application Publication
(10) Pub. No.: US 2023/0149337 A1
(43) Pub. Date: May 18, 2023

(54) OPHTHALMIC FORMULATION AND ITS USE

(71) Applicant: OY FINNSUSP AB, Lieto (FI)

(72) Inventor: Jarmo LAIHIA, Lieto (FI)

(21) Appl. No.: 17/915,019

(22) PCT Filed: Mar. 30, 2021
(86) PCT No.: PCT/EP2021/058220

§ 371 Date: Sep. 27, 2022

(30) Foreign Application Priority Data
Mar. 31, 2020 (WO) 20167272.2

(51) Int. Cl. A61K 31/202; A61K 9/00; A61K 9/107; A61K 47/44; A61K 47/18; A61K 47/26; A61K 47/12; A61K 47/10; A61K 47/22; A61K 31/23; A61P 27/04; A61K 45/06

(52) U.S. Cl. CPC A61K 31/202; A61K 9/0048; A61K 9/1075; A61K 47/44; A61K 47/18; A61K 47/26; A61K 47/12; A61K 47/10; A61K 47/22; A61K 31/23; A61P 27/04; A61K 45/06

(57) ABSTRACT

The invention relates to an ophthalmic formulation in form of an oil-in-water microemulsion comprising oil particles dispersed in a continuous water phase and its uses. The formulation comprises 0.01-20.0 weight-%, preferably 0.1-2.0 weight-%, of a natural oil comprising triglycerides which comprise >50 weight-% of unsaturated fatty acid(s) with an aliphatic chain length of 16-20 carbon atoms, and at least a first non-ionic emulsifier and a second non-ionic emulsifier. Both the first non-ionic emulsifier and the second non-ionic emulsifier comprise at least one structural unit with an aliphatic chain length of 16-20 carbon atoms originating from an unsaturated fatty acid. The formulation has a surface tension in a range of 40-43 mN/m at an emulsion-air interface at a temperature of 15-30° C.

### FIELD OF INVENTION

The present invention relates to an ophthalmic formulation and its use according to the preambles of the enclosed independent claims.

### BACKGROUND

The tear film has an invaluable role for ocular health in protecting and moisturizing the corneal and conjunctival surfaces of the eye. It also forms a refracting surface for light entering the visual system. Tear film is the part of tear fluid that spreads onto the ocular surface when the eyelids are opened, thus called the preocular tear film. The structure of the preocular tear film has been traditionally described using a three-layer model: the innermost mucin layer covers and attaches to the surface of the ocular epithelium; the aqueous layer provides lubrication, nutrients, antimicrobial proteins and appropriate osmolarity to maintain the viability of the exposed ocular cells; and the outermost lipid layer prevents excessive evaporation of water from the aqueous layer. The aqueous and mucin layers have been later commonly considered to form a single mucoaqueous layer instead of two distinct layers.

The tear film lipid layer is derived from excretions of the meibomian glands at the lid margins and is spread onto the tear film with each blink. Certain physical and rheological characteristics, such as surface tension, viscosity, pH and osmolarity, dictate the ability of the tear film to establish a stabile protective and moisturizing layer onto the ocular surface after spreading. Defects in these key characteristics may lead to impairment of tear film function, which may manifest as ocular surface disorders including, but not limited to, dry eye disease, blepharitis, Stevens-Johnson syndrome, Sjőgren's syndrome, ocular cicatricial pemphigoid and graft-versus-host disease.

Dry eye is the most common ocular surface disorder and these terms are often even used interchangeably. Dry eye can be caused by multiple intrinsic, environmental or iatrogenic factors. It is known that hyperosmolarity and elevated surface tension are found in tears of patients with dry eye, leading to desiccation-induced stress and death of ocular epithelial cells. The physiological surface tension in the tears of healthy individuals is reported to be in the range 38-46 mN/m. In tears from patients with dry eye, the values have increased close to 50 mN/m; simultaneously, the spreading of the tear film is remarkably retarded. Elevation of both osmolarity and surface tension may result from a defective tear film lipid layer. In addition, instability of the preocular tear film, observable as a short tear film break-up time representing the retention time of tear film on the surface of the open eye, is a consequence of poor quality and slow spreading of the tear film during blink cycles. These disturbances in the tear film lead to epithelial damage, sensations of uncomfortable or painful ocular symptoms and impaired visual function.

Typical first-line treatment options for dry eye include topically administered ocular lubricants such as eye drops, gels and sprays. The ultimate aim of dry eye management is to restore homeostasis of the ocular surface and to prevent a return to the vicious cycle of dry eye. Lipid-containing eye drops have the advantage to support the tear film lipid layer. They can often be formulated as various types of dispersions, such as emulsions. Preferably the emulsions used as ocular lubricants should be optically transparent, stable and homogenous, to avoid blurring of vision and requiring no shaking or inverting of the dispensing bottle to restore homogeneity of the emulsion prior to application to the eye.

Laihia et al. (Acta Ophthalmol. 2019 Oct. 3. doi:10.1111/aos.14252) discloses the results of a randomized clinical trial in which patients with dry eye were treated with microemulsion eye drops (a non-commercial investigational product) comprising 0.1% sacha inchi (Plukenetia volubilis) seed oil. The ophthalmic formulation was optically clear and stable. The authors suggest that the observed improvement in ocular surface protection by the tear film resulted from strengthening of the tear film lipid layer and lowering of its surface tension. However, there is no indication about the surface tension of the formulation used.

Furthermore, there is a general interest to topically administer medicinal and non-medicinal active agents to the eye or on eyelids. It is known that various anatomical, physiological and physico-chemical barriers significantly weaken the bioavailability of active agents from conventional drug delivery systems. For example, the poor ocular bioavailability of drugs from conventional eye drops due to the physiological barriers of the eye can be improved by incorporating the drug into emulsion-based drug delivery systems that improve precorneal retention and ocular permeation. The disadvantages of these systems include local side effects such as ocular pain and poor stability of the product under storage. Consequently, there is a need for improved ways to topically administer active agents, especially to the eye or on eyelids.

### SUMMARY OF THE INVENTION

The object of the present invention is to minimize or even eliminate the disadvantages existing in the prior art.

An object of the present invention is to provide an ophthalmic formulation that is optically transparent, thermodynamically stable and with a physiological surface tension which corresponds to healthy human tears.

A further object of the present invention is to provide an ophthalmic formulation that is suitable for treatment or prevention of ocular surface disorders, especially exemplified by impaired tear film stability, impaired tear film retention and/or impaired tear film spreading, or by tear film hyperosmolarity.

All the described embodiments and advantages apply to all aspects of the present invention, both the formulation and its uses, whenever applicable, even if not always explicitly stated so.

These objects are achieved by the features disclosed in the independent claims and the invention is defined by the features of the enclosed independent claims. Some preferable embodiments of the present invention are presented in the dependent claims.

Typical ophthalmic formulation in form of an oil-in-water microemulsion according to the present invention comprises oil particles dispersed in a continuous water phase, the formulation comprising
0.01-20.0 weight-%, preferably 0.1-2.0 weight-%, of a natural oil comprising triglycerides which comprise 50 weight-% of unsaturated fatty acid(s) with an aliphatic chain length of 16-20 carbon atoms, and
at least a first non-ionic emulsifier and a second non-ionic emulsifier, where both the first non-ionic emulsifier and the second non-ionic emulsifier comprise at least one structural unit with an aliphatic chain length of 16-20 carbon atoms originating from an unsaturated fatty acid,
wherein the formulation has a surface tension in a range of 40-43 mN/m at an emulsion-air interface at a temperature of 15-30° C.

Typical formulation according to the present invention is for use as a medicament.

Typical formulation according to the present invention is for use in a treatment of an ocular surface disorder.

Now it has been surprisingly found that an ophthalmic formulation comprising oil-in-water microemulsion according to the present invention has a surface tension in the range of 40-43 mN/m. It is assumed, without wishing to be bound by a theory, that within this surface tension range the ophthalmic formulation is capable of effectively counteracting the adversely elevated surface tension of tear fluid in patients with an ocular surface disease, such as dry eye. The surface tension in the range of 40-43 mN/m represents a specific selected sub-range within the physiological range 38-46 mN/m present in the tear fluid of healthy non-dry-eye individuals. The ophthalmic formulation according to the invention unexpectedly enhances the retention of the tear film and the protecting effect of the tear film on the ocular surface. Furthermore, the ophthalmic formulation according to the invention may decrease, to a clinically meaningful extent, the tear film hyperosmolarity, and the damage and inflammation on the ocular epithelium, all of which are currently acknowledged core etiologic factors of the vicious cycle of dry eye. By correcting, with the aid of the inventive formulation, the surface tension of the tear film of dry eye patients, the spreading of the tear film is enhanced, which improves the physiological protecting function of the preocular tear film. These advantageous effects together result in the recovery of a healthy ocular surface epithelium and tear film function, with reduced symptoms.

The formulation according to the invention has a surface tension in a range of 40-43 mN/m, or 40.5-43 mN/m, at the emulsion-air interface at a temperature of 15-30° C. The surface tension of a solution may be measured by a number of techniques known in the art, including force tensiometer or du Nouy ring method, pendant drop or sessile drop methods, Wilhelmy plate tensiometer method or bubble pressure tensiometer method. The specific surface tension of the formulation of the present invention is not only advantageous for retaining and/or stabilising the tear film of the eye. The surface tension also affects the volume of emulsion drops when administered on the eye. The specific surface tension range of the formulation according to the present invention can surprisingly reduce the volume of the drops administered from a standard eye drop bottle to a desirable drop volume of about 30 μl, which corresponds to the maximal volume of liquid the conjunctival sac of the eye is able to hold.

In the present context “oil-in-water microemulsion” denotes an emulsion comprising discrete oil particles dispersed in a continuous water phase. The oil particles are discrete particles or droplets which are uniformly dispersed in the continuous water phase. The formulations can be prepared by self-emulsification techniques, known as such for a person skilled in the art, avoiding excessive energy demands and laborious processing steps in the manufacture.

The formulation according to the present invention may comprise 0.01-20.0 weight-%, preferably 0.03-15.0 weight-%, more preferably 0.05-10.0 weight-%, even more preferably 0.07-5.0 weight-% or 0.1-2.0 weight-%, of a natural oil. The natural oil suitable for the formulation of the present invention comprises primarily triglycerides, also known as triacylglycerols, which comprise ≥50 weight-%, preferably ≥70 weight-%, more preferably ≥85 weight-% of unsaturated fatty acid(s) with an aliphatic chain length of 16-20 carbon atoms. Preferably the natural oil may be a plant-based oil or fish oil. It has been observed that plant-based oils and fish oils have an unsaturated fatty acid content that has, among others, a beneficial effect on the recovery of a healthy ocular surface epithelium. Natural oils may further comprise residual amounts of diglycerides, monoglycerides, free fatty acids, sterols, pigments, glucosides, and natural antioxidants, such as vitamins.

The unsaturated fatty acid(s) with an aliphatic chain length of 16-20 carbon atoms may be monounsaturated fatty acid(s) and/or polyunsaturated fatty acid(s). “Aliphatic chain” in the present context denotes an acyclic, unbranched, and saturated or unsaturated carbon chain. Aromatic compounds are excluded.

According to one preferable embodiment of the invention, the unsaturated fatty acid may comprise one or more monounsaturated fatty acid(s), such as palmitoleic acid (C16:1), oleic acid (C18:1), paullinic acid (C20:1), or any of their mixtures. In an alternative preferable embodiment, or in addition, the unsaturated fatty acid may comprise one or more polyunsaturated fatty acid(s), such as linoleic acid (C18:2), alpha-linolenic acid (C18:3), gamma-linolenic acid (C18:3), eicosapentaenoic acid (C20:5), or any of their mixtures. According to the most advantageous embodiment, the formulation comprises natural oil, where triglycerides comprise polyunsaturated fatty acid(s) which are able to provide various health benefits.

According to one embodiment of the invention, especially the presence of plant-based natural oil is preferable in the ophthalmic formulation. Formulations that comprise one or more plant-based natural oils are advantageous for the clinical performance of the formulation. It has been observed that natural oils from several plant species may comprise a high content of polyunsaturated fatty acids which are effective antioxidants and able to support the healing process of the ocular surface. Polyunsaturated fatty acids most abundantly present in natural plant-based oils and suitable for use in the present invention are alpha-linolenic acid (C18:3), gamma-linolenic acid (C18:3) and linoleic acid (C18:2). Preferably the formulation comprises at least one or more of these polyunsaturated fatty acids.

The natural oil suitable for the formulation according to the present invention may preferably be a seed oil, more preferably a seed oil selected from canola oil, flaxseed (linseed) oil, raspberry seed oil, kiwi seed oil, candlenut tree (kukui) seed oil, borage seed oil, camelina sativa seed oil, sacha inchi seed oil, sea buckthorn seed oil, safflower seed oil, quinoa seed oil, watermelon seed oil, lime seed oil, orange seed oil, cucumber seed oil, pumpkin seed oil, strawberry seed oil, sunflower seed oil, walnut seed oil, boxthorn seed oil, evening primrose oil, noni seed oil, passion fruit seed oil, perilla seed oil, cherry seed oil, apple seed oil, black currant seed oil, currant seed oil, dog rose seed oil, cloudberry seed oil, wheat germ oil, blueberry seed oil, cranberry seed oil, bilberry seed oil, lingonberry seed oil, grape seed oil, or corn oil. Seed oils often comprise high levels of polyunsaturated fatty acids.

The formulation according to the present invention may comprise at least a first non-ionic emulsifier and a second non-ionic emulsifier, which preferably are different from each other. Both the first non-ionic emulsifier and the second non-ionic emulsifier comprise at least one structural unit with an aliphatic chain length of 16-20 carbon atoms originating from an unsaturated fatty acid. This ensures that the first non-ionic emulsifier and the second non-ionic emulsifier are structurally compatible with the unsaturated fatty acid chains of the natural oil triglycerides present in the formulation. In this manner it is possible to enhance the emulsifying efficiency of the emulsifiers and to produce the desired surface tension and oil particle size for the ophthalmic microemulsion formulation.

The first non-ionic emulsifier may have a hydrophilic lipophilic balance value, HLB value, greater than the required HLB value of the natural oil. The concept “required HLB value” is here understood, as conventional in the art, as the HLB value identified for an individual oil, necessary to give good oil-in-water emulsification. The first non-ionic emulsifier may be an ( )eyl ether or a mono-oleate, preferably polyoxyethylene sorbitan mono-oleate. An example of a suitable emulsifier is Tween® 80.

The second non-ionic emulsifier may have an HLB value smaller than the required HLB value of the natural oil. The second non-ionic emulsifier may be an oleyl ether or a mono-oleate, preferably sorbitan mono-oleate. Examples of suitable emulsifiers are Brij® 92/93, Brij® 97, Brij® 98 or Span® 80.

The formulation may comprise the first non-ionic emulsifier and the second non-ionic emulsifier in a quantitative ratio that corresponds to the required HLB value of the natural oil. The HLB values of commercially available emulsifiers are known as such from the literature or provided by their manufacturers. The required HLB value of an oil may be determined experimentally by following general procedures known in the art concerning the HLB system (ICI Americas 1984).

In one embodiment, the formulation may comprise the first non-ionic emulsifier and the second non-ionic emulsifier in a molar ratio from 1:1 to 2.8:1.

In the formulation according to the invention, the first non-ionic emulsifier and the second non-ionic emulsifier may have a total molar concentration which is 80-150%, preferably 90-140%, more preferably 95-130%, of the total molar concentration of unsaturated fatty acid(s) of the natural oil triglycerides. It has been unexpectedly observed that when the total molar concentration of unsaturated fatty acid chains of the employed emulsifiers is approximately equal to the total molar concentration of unsaturated fatty acid chains of the natural oil triglycerides, the emulsifying efficiency is improved, resulting in an optically transparent, stable and homogenous microemulsion formulation in which the surface tension is in the desired range of 40-43 mN/m.

The oil particles in the microemulsion of the present formulation may have an average hydrodynamic diameter of 100 nm or less, preferably 50 nm or less, more preferably 30 nm or less. The particle size distribution and the average hydrodynamic diameter of emulsion particles may be determined with dynamic light scattering techniques, such as by using the Zetasizer Nano ZS instrument (Malvern Instruments, Malvern, UK). According to one embodiment, the average hydrodynamic diameter may be in the range of 0.5-100 nm, preferably 0.8-50 nm, more preferably 1-30 nm. Due to the small particle size, the formulations may be sterile filtered by conventional filtration methods and aseptically filled into various kinds of pre-sterilized primary packages, thus avoiding possible degradation processes in the formulation or in the package materials during autoclaving at high temperature and pressure. The small particle size of the oil particles also improves the stability as well as the transparency of the formulation.

The formulation may have an optical absorbance less than 0.3 absorbance units for a 1-cm path length in a wavelength range of 400-700 nm, when measured from an undiluted sample of the microemulsion formulation in a quartz cuvette. In practice this means that the formulation is optically transparent and reduces the risk for blurring of vision when the formulation is administered into the eye.

According to one embodiment of the invention, the ophthalmic formulation may further comprise at least one co-emulsifier, such as glycerol. Glycerol may augment the formation of the transparent and stable microemulsion formulation. Glycerol may also provide beneficial health effects on the ocular surface.

According to one embodiment of the invention, the oil particles may have a negative zeta potential, preferably from −100 mV to −0.5 mV, more preferably from −50 mV to −5 mV. Emulsion particles with a negative zeta potential will repel each other, preventing flocculation and stabilizing the microemulsion. Furthermore, a negative zeta potential of the oil particles may advantageously reduce or even completely eliminate the sensation of pain in the eye which is a commonly reported side effect associated with the use of emulsion formulations with a positive zeta potential. It is assumed that the oil particles with a negative zeta potential are repelled by the negatively charged eye surface epithelium, which may avoid painful sensations associated with binding to the ocular epithelium. On the contrary, the negative zeta potential of the oil particles may promote their integration into the outermost lipid layer of the tear film. In this manner, the formulation is also able effectively to counteract the adversely elevated surface tension of the tear fluid in patients with an ocular surface disease, such as dry eye.

According to one preferable embodiment, the formulation is free of organic solvents and/or preservatives. As the microemulsion may be prepared without any use of organic solvents, such as chloroform, dichloromethane, tetrahydrofuran or ethanol, the occupational safety at the manufacturing site is improved. Furthermore, as the formulation does not contain any residual amounts of organic solvents and/or preservatives, the risk for toxicity to ocular epithelial cells and allergic reactions is reduced.

The continuous water phase of the microemulsion may contain a buffering agent, such as phosphate, borate, trometamol, citrate or any of their combinations. The buffering agent is selected in view of clinical safety of the buffering agent itself when used in the eye and the desired pH range of the formulation. Typically, the pH of the formulation suitable for topical ocular administration may be in the range of 6.0-8.0, preferably 6.8-7.7. The pH of the formulation is thus within the physiologic pH range of 5.2-8.6 found in the tear fluid of healthy eyes, and the pH range of 3-8.6 tolerated by the human eye.

The formulation according to the present invention is suitable for use as a medicament. Especially the present formulation is suitable for use in the treatment of an ocular surface disorder. The ocular surface disorder may comprise impaired tear film stability, impaired tear film retention and/or tear film spreading, or tear film hyperosmolarity. The formulation according to the present invention is especially suitable for use in the treatment of an ocular surface disorder selected from dry eye disease, blepharitis, Stevens-Johnson syndrome, Sjőgren's syndrome, ocular cicatricial pemphigoid or graft-versus-host disease.

According to one embodiment the invention comprises a method for treating an ocular surface disorder comprising administrating the formulation according to the present invention to the eye or on the eyelid.

It is further possible to incorporate one or more active agents into the formulation, preferably into the oil particles of the microemulsion of the formulation, if desired. According to one embodiment the invention the formulation may comprise one or more active agents. The ophthalmic formulation according to the present invention may be used, for example, as a drug delivery vehicle, which significantly enhances the bioavailability and the penetration of active agent(s) into a biological target tissue. In the present context, the term “active agent” is understood as any substance or compound, which is capable of treating or preventing diseases or enabling a medical diagnosis in a living subject, preferably a mammal, including both humans and animals, and which is suitable for topical administration, especially when incorporated into the present formulation. According to one preferable embodiment of the invention, the active agent can be any substance or compound intended to exert a pharmacological, immunological or metabolic action with an intent of restoring, correcting and/or modifying physiological functions of the living subject or to make a medical diagnosis. According to another preferable embodiment of the invention, the active agent is non-medicinal active agent, which is intended to exert an action other than pharmacological, immunological or metabolic action for a therapeutic or diagnostic purpose.

The active agent suitable for the incorporation into the oil particles of the microemulsion of the ophthalmic formulation may include natural or synthetically produced substances and/or compounds. Natural substances and compounds may comprise, for example, substances obtained from whole, fragmented or cut plants, plant parts, algae, fungi, lichen, and/or exudates in any form and by any process. Synthetically produced substances and compounds comprise chemicals produced by routes of synthesis in organic and inorganic chemistry.

According to one preferable embodiment, the ophthalmic formulation may comprise one or more hydrophobic active agents. Hydrophobic refers here to water-repelling substances and compounds that are poorly soluble in aqueous solutions. The ophthalmic formulation according to the present invention is especially suitable for the incorporation of hydrophobic active agents, because they are readily soluble in the oil particles of the microemulsion and can be incorporated in them. The hydrophobic active agent localizes itself preferentially in the hydrophobic oil particle of the microemulsion, whereby its impact on surface properties of the oil particle or the bulk properties of the microemulsion is minimized. A balance concentration may be obtained between the fraction of the hydrophobic active agent incorporated in the oil particles and the fraction of the hydrophobic active agent present in the continuous water phase, depending on the hydrophobicity of the active agent and the concentration of the oil in the formulation. This balance can be adjusted e.g. by selection of the active agent and/or the oil concentration. Furthermore, in the oil particle the hydrophobic active agent is stabilised and protected against degradation by possible chemically reactive agents, such as oxygen radicals. The oil particles of the ophthalmic formulation are thus able to provide a long-lasting and steady release of the active agent in an intact form to exert its therapeutic or diagnostic action in the target tissue.

An ophthalmic formulation comprising a hydrophobic active agent may be manufactured without any modifications in the manufacturing process and without significantly affecting the physical and chemical properties of the obtained ophthalmic formulation, such as its storage stability. For example, a hydrophobic active agent may be first dissolved in the natural oil which is then used for the manufacture of the microemulsion of the ophthalmic formulation. Alternatively, it is possible to first manufacture the microemulsion of the ophthalmic formulation; the active agent is then added to the microemulsion, where it is gradually dissolved and incorporated into the oil particles of the microemulsion. The final concentration of the active agent in the ophthalmic formulation may be easily adjusted to a desired level by changing either the concentration of the active agent dissolved in the oil or the concentration of the oil in the ophthalmic formulation.

Active agent(s) known in the field of ophthalmology and suitable for incorporation into the microemulsion of the ophthalmic formulation may be selected from—anti-infective agents, i.e. antimicrobial, antiviral and/or antiprotozoal agents, such as aciclovir, azithromycin, cefuroxime, chloramphenicol, chlortetracycline, fluoroquinolones, fusidic acid, ganciclovir, natamycin, propamidine, and tobramycin;
corticosteroids, such as dexamethasone, fluocinolone, acetonide, fluorometholone, hydrocortisone, and prednisolone;
anti-inflammatory analgesics, such as bromfenac, diclofenac, ibuprofen, ketorolac, nepafenac, oxycodone, paracetamol, tramadol, and triamcinolone;
glaucoma medications, such as apraclonidine, acetazolamide, acetylcholine, betaxolol, bimatoprost, brimonidine, brinzolamide, dorzolamide, latanoprost, pilocarpine, tafluprost, timolol, and travoprost;
mydriatic and cycloplegic drugs, such as atropine, cyclopentolate, phenylephrine, scopolamine, and tropicamide;
antiallergic drugs, such as azelastine, emedastine, sodium cromoglycate, ketotifen, levocabastine, lodoxamide, and olopatadine;
local anaesthetics, such as lidocaine, oxybuprocaine, proxymetacaine, and tetracaine;
diagnostic agents such as fluorescein; and/or
immunomodulators; such as cyclosporine.

The ophthalmic formulation may comprise one or more active agents.

According to one embodiment of the invention, the formulation may be administered topically in form of eye drops, eye gel, eye ointment, eye wash, eye mist, eyelid wipe or eye spray. The formulation is suitable for topical administration to the eye or on the eyelid. Especially the formulation according to the present invention is suitable for use in the treatment of an ocular surface disorder, where the formulation may be administered topically in form of eye drops, eye gel, eye ointment, eye wash, eye mist, eyelid wipe or eye spray.

More generally the formulation according to the present invention is suitable for administration on a mucous membrane, e.g. on nasal mucous membrane.

The optically clear and thermodynamically stable formulations according to the present invention can be filled, if desired, in transparent or translucent primary packages and used without the need for shaking, thus increasing the commercial appeal. This means that the present formulation is optically transparent, stable and homogenous, requiring no restoration of the homogeneity of the formulation prior to application.

### BRIEF DESCRIPTION OF THE DRAWINGS

FIGS. 1A-1F show clinical efficacy of SIME02A, denoted as SIME, and HA control eye drops for signs of dry eye.

FIG. 1A: Ocular protection index (OPI); mean±SD (ITT population, n=26 per group). Dash line marks OPI=1.

FIG. 1B: Tear osmolarity in patients with baseline tear hyperosmolarity (≥308 mOsm/kg, dash line); mean±SD (ITT population, n=8 for SIME, n=14 for HA).

FIG. 1C: Corneal staining; mean±SD of the mean values of eyes (Oxford scale 0-5) (PP population, n=24 for SIME; n=25 for HA).

FIG. 1D: Nasal conjunctival staining; mean±SD of the mean values of eyes (Oxford scale 0-5) (PP population, n=24 for SIME; n=25 for HA).

FIG. 1E: Conjunctival redness; mean±SD of the mean values of eyes (IER scale 0-4) (safety/ITT population, n=26).

FIG. 1F: Lid redness; mean±SD of the mean values of eyes (IER scale 0-4) (safety/ITT population, n=26).

FIGS. 2A-2D show box-whisker plot of ocular surface disease index (OSDI). The mean is marked with a diamond. Boxes represent interquartile ranges separated by the median; whiskers indicate the minimum and maximum values (ITT population, n=26). ***, p<0.0001.

FIG. 2A: OSDI sum score.

FIGS. 2B-2D: Subscale scores of OSDI.

FIG. 3 shows UV-VIS absorption spectrum of microemulsion SIME04 with sacha inchi seed oil. No significant absorption in the visible wavelength range 400-700 nm is observed in emulsion solutions stored (A) at room temperature (about 20-25° C.) or (B) at about 45° C. for 3 months.

FIG. 4 shows correlation of pH and UV absorption (at 270 nm) of microemulsion SIME04.

FIG. 5 shows stability analysis of microemulsion SIME02A, mean±SD, n=3. SD bars may not be visible due to their small values.

FIG. 6 shows hydrodynamic diameter (A) and zeta potential (B) distribution of microemulsion SIME02A stored at room temperature (22° C.) and at 45° C.

FIGS. 7A-7E show surface pressure (FIG. 7A), surface potential isotherms (FIG. 7B) and the average intensities of the Brewster angle microscopy (BAM) images (FIG. 7C) of sacha inchi (SI) seed oil and SIME02A in a Langmuir trough system. FIG. 7D illustrates surface pressure isotherms of 20-OAHFA film with various amounts of SIME02A added to the system, and FIG. 7E shows BAM images of 20-OAHFA lipid films, captured at time points corresponding to isotherm locations marked in FIG. 7D with letters [a] to [d].

FIG. 8 shows hydrodynamic diameter and zeta potential and their stabilities in microemulsions loaded with dexamethasone (DXM). Microemulsions were stored for 3.5 months at room temperature of about 22° C. or at temperature of about 32° C. Compositions of DXM-loaded microemulsions EE27 (A, C) and EE26 (B, D) are shown in Table 4; corresponding reference samples without DXM (dash curve lines) are with an oil concentration of 0.1% (A) and 0.5% (B).

FIGS. 9A-9D show the release kinetics of dexamethasone (DXM) as detected in receiver compartments of RED inserts; mean±SD from 3 independent experiments each.

FIG. 9A: the relative cumulative release of DXM from pre-loaded microemulsions EE27 and EE26 (1 mg/ml DXM).

FIG. 9B: transmission of DXM dissolved in the corresponding buffer solutions (0.1 mg/ml DXM).

FIG. 9C: the release from DXM-spiked and DXM-pre-loaded microemulsion EE26, both with 0.1 mg/ml DXM.

FIG. 9D: the release from DXM-spiked and DXM-pre-loaded microemulsion EE27, both with 0.1 mg/ml DXM.

FIG. 10 shows the cumulative amount of dexamethasone (DXM) permeated across the porcine cornea from DXM-loaded (1 mg/ml) microemulsions EE27 and EE26, from a commercial lipophilic eye ointment (1 mg/ml), and from aqueous BSS Plus buffer solution (0.1 mg/ml) during six hours; mean±SEM from 3 or 4 independent experiments.

### DETAILED DESORPTION OF THE INVENTION

Some embodiments of the invention are described in the following non-limiting examples.

### Example 1

A series of microemulsion formulations was prepared according to Table 1. All chemicals are generally available from various commercial suppliers. The formulation comprised sacha inchi seed oil (natural oil), Tween® 80 (the first non-ionic emulsifier) and Span® 80 (the second non-ionic emulsifier), and glycerol (co-emulsifier). The needed concentration of the first and second non-ionic emulsifier to emulsify a certain percentage of oil was determined according to general instructions concerning the HLB system (ICI Americas 1984) by using spectral absorption in the UV/visible wavelength range as an endpoint. The HLB values for Span® 80 and Tween® 80 are known from the literature. The most optimal concentrations were those producing the least absorption. Fine-tuning of the concentrations by use of mathematical fitting procedures for the absorption data resulted in the required HLB value for the oil. Next, the required minimum total amount of both emulsifiers per the amount of oil in the obtained HLB ratio were subsequently determined. As the last step, the required minimum concentration of the co-emulsifier was determined as well. After these adjustments, the relative concentrations of oil, emulsifiers and co-emulsifier are known, and the microemulsion composition has the lowest possible absorbance (maximal transmittance) and visual transparency. At each step explained, the lipid component was mixed by using a conventional magnetic stirrer with heating to above about 50° C. The aqueous buffered solution containing sodium hyaluronate, KCl and trehalose was gradually added until the desired oil concentration was reached. Without any further adjustments, the surface tension was measured using Krűss K6 force tensiometer by the ring method (Krűss GmbH, Hamburg, Germany) at room temperature. A control solution CS01 was prepared without a lipid component for reference.

The results presented in Table 1 suggest that the lipid component comprising natural oil contributes to lowering of the surface tension. Even up to 5-fold changes (0.1%→0.5%) in the concentration of the natural oil does not shift the surface tension outside the 40-43 mN/m range. It can also be seen in Table 1 that the drop volume obtained from a conventional eye drop bottle is affected by surface tension; the most preferable volume that the human eye can hold is about 30 μl which can be obtained with microemulsion formulations comprising the lipid component. The eye drop volume must be strictly distinguished from the phenomenon of droplet or particle size of the emulsion.

### Example 2

A microemulsion formulation SIME02A was prepared. The composition of SIME02A corresponds to the composition of SIME02 of Table 1, except that the amount of sodium hyaluronate (HA) was 0.2 weight-%. A control formulation comprised 0.2% sodium hyaluronate (HA) in isotonic phosphate-buffered saline without natural oil.

The formulations were visually similar (clear, homogenous and colourless), sterile filtered and aseptically filled into identical eye drop bottles designed for preservative-free administration, allowing complete masking of the treatments from the patients and the investigator. Fifty-two adult patients with moderate or severe dry eye, allocated into randomized SIME02A or control groups (n=26 for both), self-administered the corresponding eye drops on both eyes three times a day for about 30 days.

On Day 30, with no morning dose taken, clinical assessments were performed. All study assessments at baseline and on Day 30 were performed in an examination room monitored for temperature and relative humidity (RH). Safety and ocular tolerability, evaluated as the primary objective, included best corrected visual acuity (ETDRS charts 1 & 2, 2000 series), conjunctival (bulbar) and lid redness (IER grading for both; 0=not existing, 1=very slight, 2=slight, 3=moderate, 4=severe), intraocular pressure (IOP; Goldmann tonometer), digital photography of the anterior eye, and visual assessment of ocular reactions. Ocular biomarkers used as primary efficacy endpoints included tear osmolarity (I-PEN® Tear Osmolarity System, I-MED Pharma, Dollard-des-Ormeaux, QC, Canada) and fluorescein TBUT. For secondary efficacy objectives, blink rate and corneal and conjunctival staining (Oxford grading 0-5 for both) were assessed; blink rate (min⁻¹) was converted to interblink interval (IBI; s) to calculate the ocular protection index (OPI=TBUT/IBI). The ocular surface disease index (OSDI) was used for both safety and efficacy. Adverse events (AEs) were collected throughout the 30-day test period.

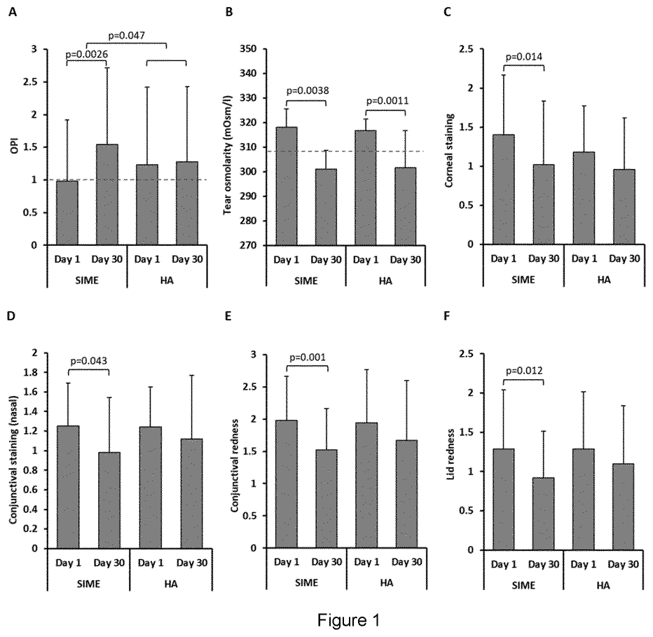

A total of 49 subjects completed the 30-day test period per protocol (PP); safety data was obtained from all 52 randomized intent-to-treat (ITT) subjects. Both formulations were well tolerated without treatment-related AEs. TBUT increased significantly by about 50% with SIME02A and non-significantly by 25% with control HA, whereas blink rates did not change considerably (Table 2). OPI improved significantly by 57% up to about 1.5 OPI units with SIME02A (p=0.0026, ANOVA) and by 4% with HA; treatment difference in changes from baseline was statistically significant for ITT population (p=0.047) and marginally significant for PP population (p=0.071) in favour of SIME02A (FIG. 1A, Table 2).

In both SIME02A group and the control group, mean tear osmolarity was initially <308 mOsm/I and showed a small decrease by Day 30 (Table 2). In hyperosmolar ITT and PP datasets, tear osmolarities reduced significantly to about 300 mOsm/I with both treatments (FIG. 1B, Table 2).

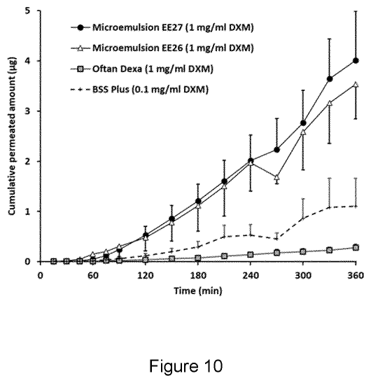

The effect of SIME02A formulation on all staining scores assessed after 30 days was greater than that of control formulation HA, although without statistical difference between treatments (Table 2). Mean ocular staining scores were of absent-to-mild intensity at baseline (0-2 score units on Oxford scale 0-5). Corneal staining decreased significantly with SIME02A in PP dataset (−27%, p=0.014, Wilcoxon signed rank test) and marginally significantly in ITT subjects (−20%, p=0.077) (FIG. 10, Table 2). Similarly, SIME02A reduced conjunctival staining with statistical significance in the nasal conjunctiva of PP subjects (−22%, p=0.043) and with marginal significance in ITT subjects (−19%, p=0.059) (FIG. 1D, Table 2).

Significantly reduced mean conjunctival (−23%, p=0.001) and lid (−29%, p=0.012) redness scores were observed with SIME02A in the safety/ITT dataset, whereas changes in the control group remained milder (ca. −15%) and non-significant (FIGS. 1E and 1F, Table 2).

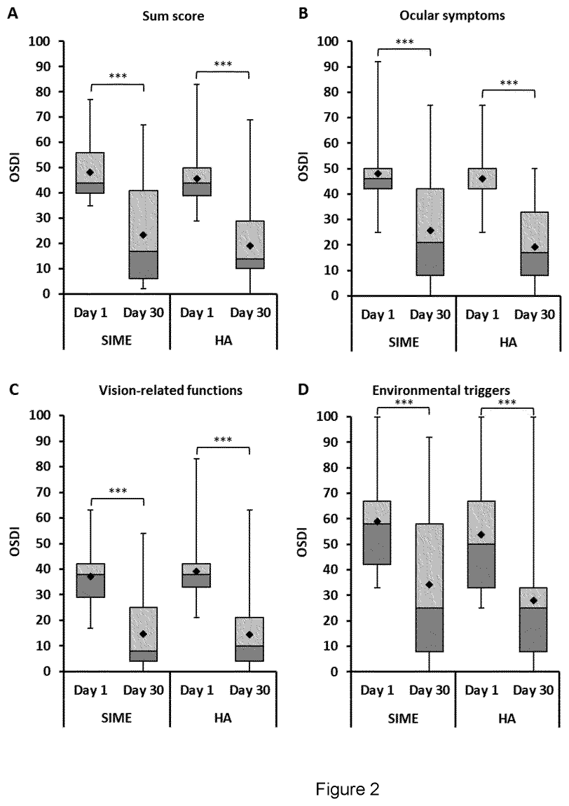

Finally, the mean OSDI sum score decreased with both SIME02A formulation (−51%) and control formulation HA (−58%) (ITT dataset; p<0.0001 for PP and ITT populations), most patients reporting mild or non-existent residual symptoms on Day 30 (medians 17 and 14, respectively) (FIG. 2, Table 2). Similarly, a highly significant reduction was seen in all three OSDI subscales (p<0.0001) (FIG. 2B-D). Of interest, the median of “vision-related functions” subscale decreased from 38 score units in both groups to units (FIG. 2C), suggesting that the majority of subjects were effectively without vision-related functional symptoms after 30 days.

The results of the study suggest that SIME02A formulation was able to improve each of the three etiologic factors for dry eye, comprising (1) tear film instability, (2) tear hyperosmolarity and (3) cellular damage and inflammation, as categorized in Table 2, while the non-lipid control treatment was able to correct hyperosmolarity only. Because the control formulation HA contained only sodium hyaluronate, it can be deduced that the lipid component comprising natural oil of SIME02A was responsible for the clinical treatment effect. The surface tension measured in a solution corresponding to the control formulation HA and additionally supplemented with 2% trehalose is 73.7 mN/m, thus, as the surface tension of the control solution CS01 is 68 mN/m (Table 1), the surface tension of the control formulation HA used in the clinical trial must be in the range 68-73.7 mN/m, i.e. in a close proximity to that of pure water (Table 1). The shown improvements in the etiologic factors were accompanied by resolution of dry eye symptoms (FIG. 2, Table 2), confirming both clinical safety and efficacy of SIME02A.

### Example 3

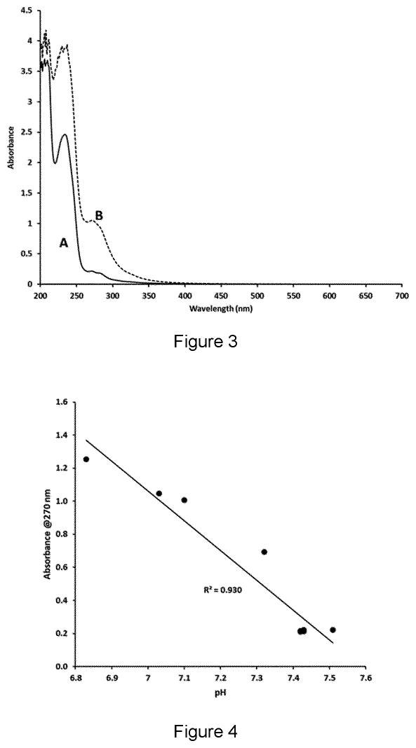

A microemulsion formulation SIME04, as described in Table 3 was prepared, sterile filtered and aseptically filled into bag-in-bottle-type eye spray containers designed for preservative-free administration. The bottles were subjected to stability testing at temperatures 22±2° C. (sample A) and 45±2° C. (sample B) for 3 months. At the end of testing period, sample B was visually clearly more turbid but still perfectly homogenous. Absorption spectra measured over 200 to 700 nm (FIG. 3) for samples A and B after 3-month storage show that very small changes take place in the visible wavelength range 400-700 nm, whereas larger differences are seen in the UV range at below 350 nm. The absorbance of sample A after 3-month storage corresponded to the initial absorbance at time of manufacture (not shown in FIG. 3), demonstrating no significant changes at 22±2° C.

In a further step, samples of microemulsion formulations SIME04 were stored for various time periods in different conditions and analysed for pH and absorbance at 270 nm. The wavelength 270 nm was selected based on a large absorbance change at that wavelength, as shown in FIG. 3. The results are shown in FIG. 4 where both parameters are plotted in the same graph. It can be seen that there is a strong correlation between the pH of the solution and its UV absorbance. This observation reveals that pH can be used as a convenient proxy parameter for stability analyses of microemulsions.

### Example 4

Three separate batches of a microemulsion formulation SIME02A as in Example 2 were prepared, sterile filtered and aseptically filled into gamma-sterilized semi-transparent eye drop bottles designed for preservative-free administration. Sample bottles from these batches were subjected to stability testing at temperatures 22±2° C. and 32±2° C. pH was used as a proxy parameter for changes in the microemulsion formulation according to Example 3. The results presented in FIG. 5 indicate that microemulsion formulation SIME02A was stable for the studied time period of up to 18 months at both temperature conditions; pH did not significantly decrease, corresponding to visual transparency which was similar to the initial microemulsion with no visible turbidity, particles, separation of phases, or colour changes.

### Example 5

Microemulsion formulation SIME02A as in Example 2 was prepared. The hydrodynamic size distribution of the microemulsion particles were measured with dynamic light scattering (Zetasizer Nano ZS instrument, Malvern Instruments, Malvern, UK) using 173° back-scattered light signal at 25° C. The hydrodynamic diameter of the particles was calculated using the intensity-weighted Stokes-Einstein equation. Zeta potential measurements were performed with electrophoretic light scattering (Zetasizer Nano ZS) at 25° C.

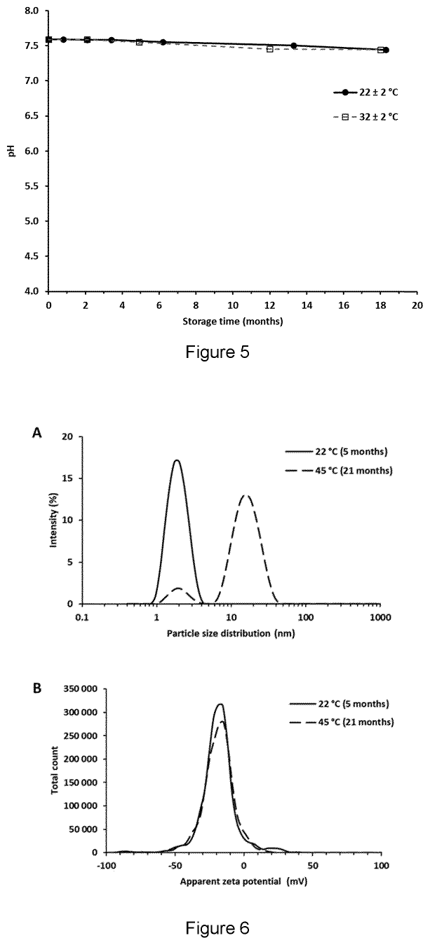

The obtained particle size distribution presented in FIG. 6 shows that the average hydrodynamic diameter was 1.4±0.1 nm in samples stored at 22° C., indicating a transparent and monodisperse microemulsion with very small particles. After storage for 21 months at 45° C., the majority of emulsion particles were about ten times larger, while a small part retained their original size (FIG. 6A), still indicating a transparent microemulsion. The average zeta potential of the particles in the same samples was −19±1 mV at 22° C. and −18±2 at 45° C., demonstrating practically unchanged zeta potential during storage at a high temperature (FIG. 6B).

### Example 6

The interactions of the microemulsion formulation SIME02A explained in Example 2 with a tear film lipid layer (TFLL) component were studied in a Langmuir trough model system. A naturally occurring lipid 20 (oleoyloxy)icosanoic acid (20-OAHFA) which represents the O-acyl-ω-hydroxy fatty acids in the TFLL was selected as a model lipid and synthesized as described in Bland et al. Langmuir 2019, 35, 3545-3552. The experiments were performed on a KSV NIMA Large trough equipped with a surface pressure balance with a platinum Wilhelmy plate, a KSV SPOT surface potential meter and KSV NIMA microBAM Brewster angle microscope (BAM), all from Biolin Scientific (Stockholm, Sweden). The Langmuir trough system was enclosed in a plastic hood with an inlet supply of dry and ozone-depleted air (Ozone solutions ODS-3P, Hull, Iow. USA) to prevent oxidation of lipids.

The Langmuir trough was filled with phosphate-buffered saline (PBS) as a subphase maintained at 35±1° C. to emulate the aqueous layer of the tear film. The model lipid 20-OAHFA or pure sacha inchi (SI) seed oil was dissolved in chloroform and spread on the surface of the PBS subphase. Chloroform was allowed to evaporate for 5 min. After formation of the lipid film, SIME02A was added in volumes of 1-20 μl. The lipid film was compressed and expanded in the trough with two Delrin barriers at a rate of 13 mm/min while the surface pressure and surface potential were measured. The film was imaged using BAM, and the square root of BAM image intensity was used as an approximation of the relative film thickness. For the calculation of mean molecular areas, an average molecular mass of 877 g/mol was estimated for the seed oil; for SIME02A, the total number of the emulsifier and oil molecules were used.

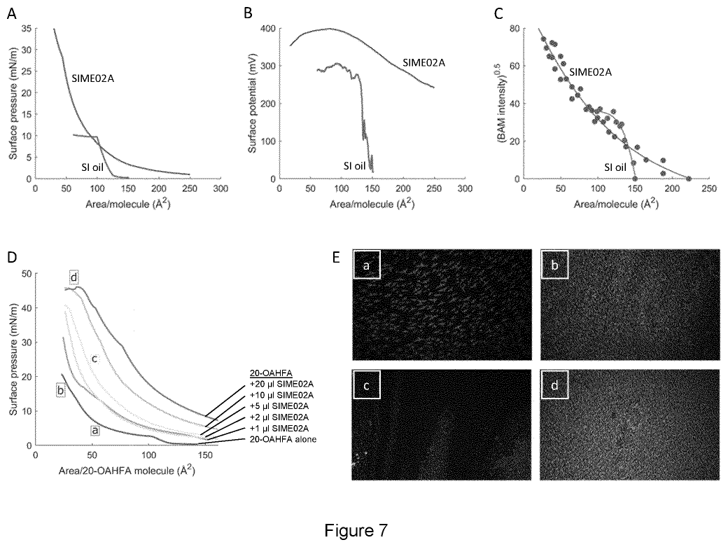

FIGS. 7A-7C show surface pressure (FIG. 7A), surface potential isotherms (FIG. 7B) and the average intensities of the Brewster angle microscopy (BAM) images (FIG. 7C) of sacha inchi (SI) seed oil and SIME02A in a Langmuir trough system. The surface pressure and surface potential isotherms show that the behaviour of SIME02A differs significantly from pure SI oil. As seen in FIG. 7A, the surface pressure isotherm of SI oil increases at approximately 125 Å²/molecule and collapses to a plateau at 10 mN/m, resembling isotherm shapes observed commonly for nonpolar triglycerides with a low surface activity. In FIG. 7C, the square root (BAM intensity)^{0.5} represents an approximation of the relative film thickness. It is seen that the surface potential (FIG. 7B) and the BAM intensity (FIG. 7C) remained fairly constant during compression after an initial increase, indicating no significant reorganization in the SI oil film before the collapse. In contrast, the microemulsion formulation SIME02A showed a higher surface activity, the surface pressure increasing already at 250 Å²/molecule (FIG. 7A). The lipid film was initially very thin compared to the SI oil monolayer, suggesting a very low density of hydrocarbon chains at the surface; upon compression, the film thickness increased, reaching a thickness approximately two times higher than that of SI oil film (FIG. 7C). No formation of lipid aggregates on the film surface were observed with SIME02A in BAM images (not shown).

FIG. 7D illustrates surface pressure isotherms of 20-OAHFA film with various amounts of SIME02A added to the Langmuir trough system; BAM images of 20-OAHFA lipid films shown in FIG. 7E were captured at time points corresponding to isotherm locations marked in FIG. 7D with letters [a] to [d]. The pure 20-OAHFA film spreads as a liquid monolayer on the subphase and undergoes transition to a solid monolayer under compression at 2.5 mN/m surface pressure (FIG. 7D[a]) and appearing as crystalline domains in BAM (FIG. 7E[a]). Addition of SIME02A to the film caused the isotherms to shift towards higher surface pressures, indicating that the microemulsion molecules readily integrate into the lipid film. Overall, the addition of 10-20 μl of SIME02A caused the isotherms (FIG. 7D, two uppermost lines) to resemble that of pure SIME02A (FIG. 7A, blue line). The surface-active molecules in 1-20 μl of added SIME02A correspond to approximately 6-130% of 20-OAHFA molecules. Inspecting these isotherms alone, the surface organization seems to be highly altered by the addition of the microemulsion. Interestingly, however, the BAM images show the 20-OAHFA film structure to remain largely unaffected even by the highest amount of SIME02A added to the film; the solid-phase structure (FIG. 7E[c, d]) forms similarly to pure 20-OAHFA (FIG. 7E[b]). The addition of SIME02A to 20-OAHFA films did not significantly affect the evaporation resistance of the film (data not shown).

The results of Example 6 uncover two main findings. Firstly, the microemulsion formulation SIME02A readily integrates into the lipid layer of tear film with natural 20-OAHFA. Secondly, the microemulsion does not disturb the formation of solid TFLL structures even at high surface pressures, whereby the evaporation resistance of the TFLL remains intact and undisturbed.

### Example 7

The effect of a hydrophobic active agent incorporated into the microemulsion the physical and chemical properties including storage stability was investigated. Dexamethasone (DXM) was selected as a model agent, as it is a conventional anti-inflammatory pharmaceutical and a hydrophobic synthetic glucocorticoid. Dexamethasone (Item No. 11015; Cayman Chemical, Ann Arbor, Mich., USA) was dissolved in pure sacha inchi seed oil in a concentration of 5% with mixing by a magnetic stirrer and mild warming. After complete dissolution, the DXM-oil mixture was used for the preparation of DXM-containing microemulsion by following the same protocol as used in the preceding Examples. Two alternative aqueous buffer compositions were used for microemulsions EE26 and EE27, see Table 4. The target concentrations for DXM (0.1%) and oil (2.0%) in the microemulsions were reached by pre-calculated quantities of the lipid and buffer components.

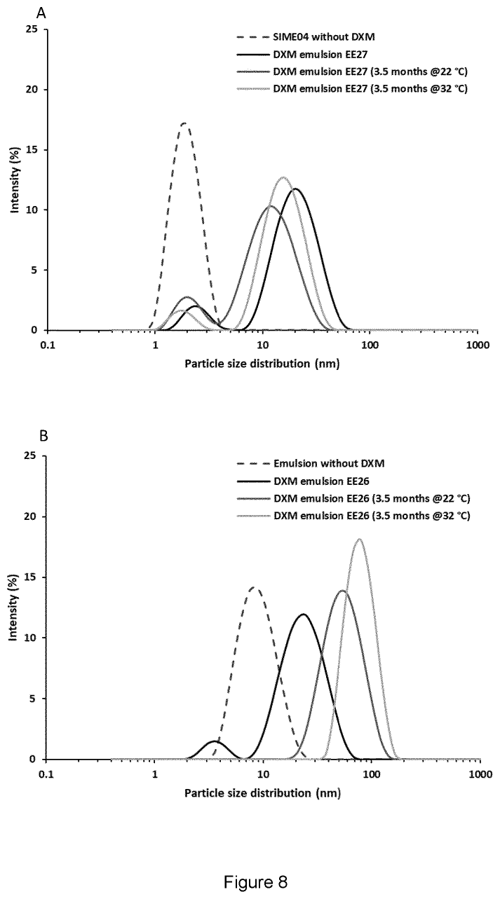

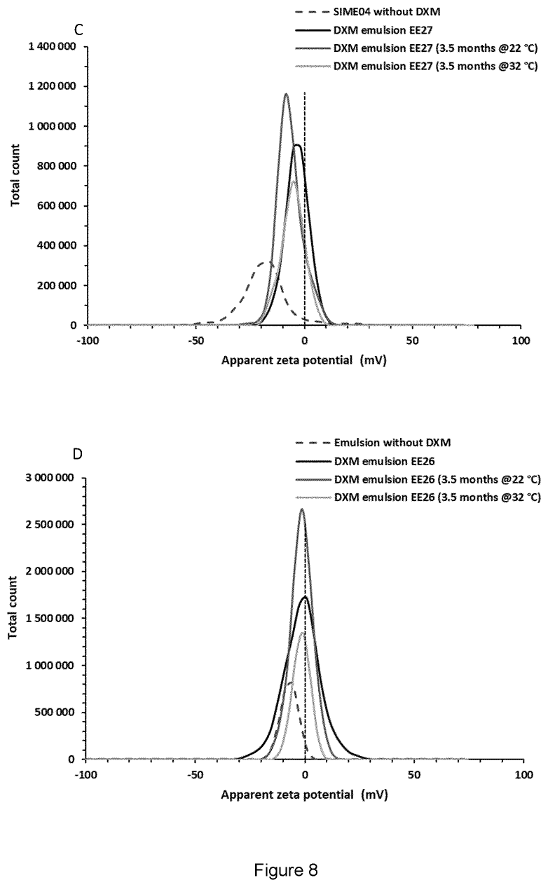

It can be seen from Table 4 that the initial pH was slightly lower and the mean hydrodynamic diameter of the emulsion particles was slightly larger for microemulsions EE26 and EE27 with DXM than in emulsions without an active agent (see Example 5). It is seen from FIG. 8 that the DXM-loaded microemulsions EE26 and EE27 showed good stability for at least three months at 22° C. and at 32° C., as the pH and the hydrodynamic diameter did not significantly change. The microemulsions EE26 and EE27 were initially transparent and did not lose this property during storage of 3.5 months. DXM did not precipitate from the microemulsions either, indicating that the active agent remained inside the emulsion particles during storage at these temperatures.

### Example 8

The solubility of dexamethasone (DXM) in the microemulsion formulations was studied. To formulations containing 0.1%, 0.5%, 1%, or 5% of the sacha inchi seed oil, DXM (10 mg/ml stock in dimethyl sulfoxide, DMSO) was added at concentrations of 5.1-6.5 mg/ml in triplicate. Phosphate-buffered saline (PBS) served as a non-emulsion control with 2.1-2.7 mg/ml of added DXM due to low solubility. The test vials were incubated at room temperature with continuous shaking (90 rpm) for 8-9 days until equilibrium. After incubation, the samples were mixed 1:1 with acetonitrile for 10 min and centrifuged (13,000 rpm, 10 min), followed by immediate HPLC analysis of the supernatant for DXM concentration. HPLC analysis was performed with Agilent 1100 series HPLC system and Eclipse XDB-C18 column (1.8 μm, 4.6×50 mm; Agilent) maintained at 60° C. Isocratic elution of the 5-μl sample injected was performed with 65% Eluent A (1% TFA in Milli-Q water) and 35% Eluent B (0.1% TFA in acetonitrile) at flow rate 1 ml/min and UV detection at 240 nm.

The solubility of DXM correlated with the oil concentration in the microemulsion, being the highest with 5% oil (ca. 3700 μg/ml). This was about 15 times higher than the solubility in PBS alone (ca. 250 μg/ml), whereas DXM solubility in the 1% oil concentration in the formulation was 2.5 times higher than in PBS. The solubilities of DXM in microemulsions with lower oil concentrations were at the same level as in PBS. The results suggest that a hydrophobic agent gradually dissolves in the microemulsion according to the oil concentration, apparently because the volume of the emulsion particles correlates with the oil concentration, making it possible to load microemulsion particles with the active agent also after preparation of the microemulsion.

### Example 9

The release of DXM from microemulsion particles was studied by using Rapid Equilibrium Dialysis (RED) inserts (ThermoFisher Scientific) in which two compartments are separated by a dialysis membrane with a size exclusion limit of 8 kDa. Samples (0.5 ml) of microemulsions EE26 and EE27 (see Table 4) with 0.1% (1 mg/ml) DXM or their 1:10 dilutions in the corresponding buffer solutions were placed into the donor side of the RED insert and the corresponding buffer (0.75 ml) into the receiver side. In a control protocol, pre-formulated control microemulsions with 2% oil (corresponding to EE26 and EE27 but without DXM) were spiked to a final concentration of 0.1 mg/ml DXM by adding the DXM stock solution (10 mg/ml in DMSO). Similar DXM spiking was done also to the buffer solutions. The RED inserts were incubated at 35° C., corresponding to the ocular surface temperature, with shaking (100 rpm). Aliquots of 200 μl from the receiver compartment were collected for up to 6 hours, each time replacing the collected volume by an equal volume of buffer. The aliquot samples were stored at −20° C. until analysed with HPLC (see Example 8 for analysis protocol details).

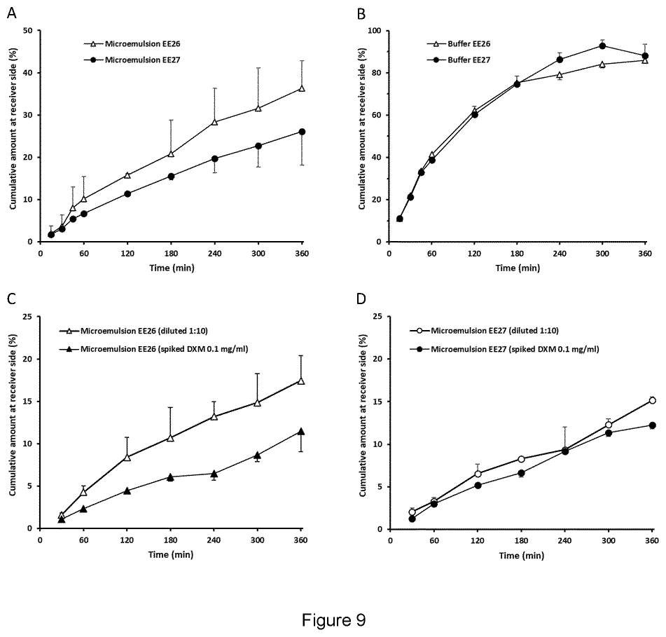

The results can be seen in FIGS. 9A-9D. Microemulsions EE26 and EE27 (1 mg/ml DXM) showed constant release rates throughout the 6-hour study (FIG. 9A). Assuming the same release rates, the duration of release until complete exhaustion would be about 16 hours for EE26 and 23 hours for EE27 as predicted by linear extrapolation. In contrast, exhaustion of DXM transmission to the receiver chamber was reached already after 4 to 5 hours in the corresponding buffer solutions (0.1 mg/ml DXM; FIG. 9B), suggesting that the ingredients and the properties of the buffer composition do not govern the transmission kinetics. DXM release from the DXM-spiked control microemulsions (0.1 mg/ml DXM) was fairly similar to the release from DXM-pre-loaded microemulsions EE26 and EE27 that were diluted to the same concentration (0.1 mg/ml DXM; FIG. 9C, D). The results indicate that the microemulsions provide extended release of DXM from the microemulsion particles at a constant rate independently of the initial DXM concentration. The duration of release is significantly longer and the transmitted amount of the active agent is significantly higher than what can be achieved with the buffer alone. Similar release kinetics are obtained either by pre-loading the active agent in the oil before preparation of the microemulsion or by dissolving the active agent afterwards in the microemulsion.

### Example 10

The effect of the microemulsion formulation on the permeability of DXM across the cornea was studied. Freshly excised porcine corneas were placed in horizontal Franz-type permeability chambers maintained at 35° C. by circulating water bath. The receiver chamber was mixed by magnetic stirring. Samples (0.5 ml) of microemulsions EE26 and EE27 (see Table 4) with 0.1% (1 mg/ml) DXM were added to the donor side and 5 ml BSS Plus buffer (Alcon) to the receiver side. A commercially available DXM-containing ointment (Oftan Dexa 1 mg/g, Santen) and 100 μg/ml DXM in BSS Plus buffer were used as controls. Aliquots of 0.5 ml were collected from the receiver chamber at several time points for up to 6 hours, replaced by an equal volume of BSS Plus buffer. The aliquot samples were stored at −20° C. until analysed with LC-MS/MS.

For LC-MS/MS analysis, the samples were filtered (Agilent Captiva ND 0.2-μm filter plate). The LC-MS/MS system consisted of an Agilent 1290 series liquid chromatograph and an Agilent 6495 triple-quadruple mass spectrometer (Agilent Technologies) with electrospray ionization. Separation of DXM and the internal standard was performed on Poroshell 120 SB-C18 column (2.1×50 mm, 2.7 μm, Agilent) maintained at 50° C. A binary mobile phase with gradient elution was used. Eluent A was milli-Q water with 0.1% formic acid and eluent B was methanol. The gradient was as follows: 0.0 to 2.0 min: 30% to 90% B; 2.0 to 2.5 min: 90% B; 2.5 to 2.6 min: 90% to 30% B; and 2.6 to 4.0 min: 30% B. The flow rate was 0.5 ml/min and the injection volume was 4 μl. Nitrogen was used as a drying, nebulizer, and collision gas. The following ion source conditions were employed: positive ion mode, drying gas temperature 200° C. and flow 16 l/min, and nebulizer pressure 25 psi. Quantification was based on selected reaction monitoring of ions m/z 393→m/z 373.1 (collision energy,CE 4) and m/z 393→m/z 355 (CE 12) for DXM, and m/z 398→m/z 378 (CE 8) and m/z 398→m/z 360.2 (CE 8) for DXM-d5. Dwell times were 100 ms. Calibration curves were determined in duplicate, and quadratic fitting with 1/×2 weighting was used.

The results are shown in Table 5 and FIG. 10. Table 5 shows the apparent permeability, Pₐₚₚ, of DXM across the porcine cornea from microemulsions EE26 and EE27 in comparison to the commercial ophthalmic ointment and the buffer solution. It can be seen that the permeability of DXM across the porcine cornea was more than 10-fold faster from microemulsions EE26 and EE27 than from the commercial ointment product. The apparent permeability (Pₐₚₚ) was even higher from the buffer solution containing 0.1 mg/ml DXM, suggesting that the hydrophobic model drug distributes more easily from the aqueous solution to the corneal tissue than from a lipophilic formulation such as an ointment. Similarly, in the microemulsions EE27 and EE26, the partition of DXM to the lipid phase most probably slows down its permeation through the cornea, creating a controlled release system, compared to permeation from the aqueous solution. However, due to a higher solubility of DXM in the microemulsion than in the buffer, the total dose of the drug permeated from the microemulsion across the cornea was much higher than from the buffer, as seen in FIG. 10. The results indicate that incorporating a hydrophobic active agent in the microemulsion both improves significantly its ocular permeation in comparison to a lipophilic state-of-the-art product and increases the total permeated dose in comparison to what can be achieved from an aqueous solution.

It is apparent to a person skilled in the art that the invention is not limited exclusively to the examples described above, but that the invention can vary within the scope of the claims presented below.

TABLE 1
Composition and properties of microemulsion formulations
(SIME), control solution (CS01) and water.

| | | | | |
|---|---|---|---|---|
| SIME01 | SIME02 | SIME03 | CS01 | Water |

Reagent

| | | | | | |
|---|---|---|---|---|---|
| Trometamol | 0.29% | 0.29% | 0.29% | 0.29% | — |
| Citric acid monohydrate | 0.14% | 0.14% | 0.14% | 0.14% | — |
| Sodium hyaluronate | 0.20% | 0.25% | 0.25% | 0.25% | — |
| Trehalose dihydrate | 2.0% | 2.0% | 2.0% | 2.0% | — |
| Potassium chloride | 0.17% | 0.17% | 0.17% | 0.17% | — |
| Sacha inchi seed oil | 0.5% | 0.1% | 0.3% | — | — |
| Tween ® 80 | 1.51% | 0.30% | 0.91% | — | — |
| Span ® 80 | 0.34% | 0.068% | 0.21% | — | — |
| Glycerol | 0.88% | 0.88% | 0.88% | 0.88% | — |
| Purified water | ad 100% | ad 100% | ad 100% | ad 100% | ad 100% |

Measurements

| | | | | | |
|---|---|---|---|---|---|
| pH | 7.57 | 7.54 | 7.55 | nd¹ | nd¹ |
| Osmolality (mOsm/kg) | 246 | 242 | 245 | 236 | 0 |
| Drop size (μl)² | 36.7 | 30.0 | 26.7 | 40.0 | nd¹ |
| Surface tension (mN/m) | 41.5 | 42.6 | 42.6 | 68.0 | 70.0 |

¹nd, not determined;
²determined by using identical eye dropper bottles for each solution

TABLE 3
Composition of microemulsion SIME04

SIME04

Reagent

| | |
|---|---|
| Trometamol | 0.29% |
| Citric acid monohydrate | 0.14% |
| Sodium hyaluronate | 0.02% |
| Sodium chloride | 0.19% |
| Potassium chloride | 0.04% |
| Sacha inchi seed oil | 0.5% |
| Tween ® 80 | 1.52% |
| Span ® 80 | 0.33% |
| Glycerol | 0.85% |
| Purified water | ad 100% |

Measurements

| | |
|---|---|
| pH | 7.51 |
| Osmolality (mOsm/kg) | 207 |

TABLE 2
Summary of results categorized by dry eye etiology, Example 2

| | | | Mean ± SD change from baseline on Day 30 | | |
|---|---|---|---|---|---|
| Etiologic | Measured | | SIME02 | HA | |
| factor and | variable | Study | ITT, n = 26; | ITT, n = 26; | Effect size; |
| symptoms | (unit) | population | PP, n = 24 | PP, n = 25 | 95% CI¹ |
| 1. Tear film | TBUT | ITT | 1.71 ± 3.42 | 0.87 ± 1.81 | 0.85; −0.68 to |
| instability | (s) | | (p = 0.0025)* | (p = 0.11) | 2.37 (p = 0.27) |
| | | PP | 1.65 ± 3.48 | 0.88 ± 1.84 | 0.77; −0.83 to |
| | | | (p = 0.0055)* | (p = 0.12) | 2.36 (p = 0.34) |
| | Blink rate | ITT | 0.02 ± 8.72 | −2.15 ± 10.2 | 2.17; −3.10 to |
| | (min⁻¹) | | (p = 0.99) | (p = 0.25) | 7.45 (p = 0. 41) |
| | | PP | −0.19 ± 9.03 | −1.96 ± 10.3 | 1.77; −3.81 to |
| | | | (p = 0.93) | (p = 0.32) | 7.36 (p = 0.53) |
| | OPI | ITT | 0.56 ± 1.08 | 0.05 ± 0.68 | 0.51; 0.0073 to |
| | (ratio) | | (p = 0.0026)* | (p = 0.78) | 1.01 (p = 0.047)* |
| | | PP | 0.53 ± 1.11 | 0.05 ± 0.69 | 0.49; −0.043 to |
| | | | (p = 0.0068)* | (p = 0.80) | 1.02 (p = 0.071) |
| 2. Tear film | Tear | ITT | −4.75 ± 16.2 | −1.77 ± 21.5 | −2.98; −13.6 to |
| hyperosmolarity | osmolarity, all | | (p = 0.21) | (p = 0.64) | 7.64 (p = 0.58) |
| | (mOsm/l) | PP | −4.73 ± 14.6 | −2.36 ± 21.8 | −2.37; −13.1 to |
| | | | (p = 0.22) | (p = 0.53) | 8.32 (p = 0.65) |
| | Tear | ITT | −17.1±10.4 | −15.1±16.6 | −1.99; −15.6 to |
| | osmolarity, ≥308 | | (p=0.0038)* | (p=0.0011)* | 11.6 (p = 0.76) |
| | (mOsm/l) | PP | −14.6±8.5 | −15.1±16.6 | 0.43; −13.7 to |
| | | | (p=0.015)* | (p=0.0010)* | 14.5 (p = 0.95) |
| 3A. Ocular | Corneal | ITT | −0.27 ± 0.78 | −0.21 ± 0.51 | −0.06 |
| surface damage | staining | | (p = 0.077) | (p = 0.070) | (p = 0.77) |
| | (Oxford scale | PP | −0.38 ± 0.70 | −0.22 ± 0.52 | −0.16 |
| | 0-5) | | (p = 0.014)* | (p = 0.070) | (p = 0.45) |
| | Conjunctival | ITT | −0.17 ± 0.51 | −0.02 ± 0.79 | −0.15 |
| | staining, | | (p = 0.13) | (p = 0.90) | (p = 0.53) |
| | temporal | PP | −0.19 ± 0.51 | −0.02 ± 0.81 | −0.17 |
| | (Oxford scale | | (p = 0.12) | (p = 0.90) | (p = 0.52) |
| | 0-5) | | | | |
| | Conjunctival | ITT | −0.23 ± 0.60 | −0.12 ± 0.65 | −0.11 |
| | staining, nasal | | (p = 0.059) | (p = 0.44) | (p = 0.73) |
| | (Oxford scale | PP | −0.27 ± 0.61 | −0.12 ± 0.67 | −0.15 |
| | 0-5) | | (p = 0.043)* | (p = 0.44) | (p = 0.59) |
| 3B. Ocular | Conjunctival | Safety/ITT | −0.46 ± 0.65 | −0.27 ± 0.65 | −0.19 |
| surface | redness | | (p = 0.001)* | (p = 0.065) | (p = 0.36) |
| inflammation | (IER grading | | | | |
| | 0-4) | | | | |
| | Lid redness | Safety/ITT | −0.37 ± 0.67 | −0.19 ± 0.55 | −0.18 |
| | (IER grading | | (p = 0.012)* | (p = 0.13) | (p = 0.30) |
| | 0-4) | | | | |
| Sum of planned primary and | | Safety/ITT | 4 | 1 | 1 |
| secondary end points | | PP | 4 | 1 | 0 |
| with significant improvement | | | | | |
| from baseline² | | | | | |
| Symptoms | OSDI sum | ITT | −24.6 ± 16.2 | −26.5 ± 14.6 | 1.9 |
| | score (scale | | (p < 0.0001)* | (p < 0.0001)* | (p = 0.66) |
| | 0-100) | PP | −25.0 ± 16.5 | −27.2 ± 14.5 | 2.2 |
| | | | (p < 0.0001)* | (p < 0.0001)* | (p = 0.62) |

¹Estimate of SIME02 vs. HA difference, 95% confidence interval for continuous variables, and between-group significance of change
²Excluding ad hoc analyses
*Statistically significant difference (p < 0.05)

TABLE 4
Microemulsion compositions with DXM.

| | |
|---|---|
| EE26 | EE27 |

Reagent

| | | |
|---|---|---|
| Trometamol | 0.29% | 0.29% |
| Citric acid monohydrate | 0.14% | 0.14% |
| Sodium hyaluronate | 0.02% | 0.2% |
| Trehalose dihydrate | 1.0% | 2.0% |
| Sodium chloride | 0.19% | 2.0% |
| Potassium chloride | 0.04% | 0.17% |
| DXM-sacha inchi seed oil mixture | 2.0% | 2.0% |
| Tween ® 80 | 6.4% | 6.4% |
| Span ® 80 | 1.4% | 1.4% |
| Glycerol | 17.6% | 17.6% |
| Purified water | ad 100% | ad 100% |

Measurements

| | | |
|---|---|---|
| pH | 7.32 | 7.25 |
| Zero-shear viscosity (mPas) | 5.7 | 15.5 |
| Hydrodynamic diameter (nm) | 17.7 ± 0.1 | 12.8 ± 0.1 |

TABLE 5
Apparent permeability (Pₐₚₚ) of DXM across porcine cornea from
formulations EE27 and EE26, a commercial ophthalmic ointment
(Oftan Dexa), and BSS Plus buffer (n = 2 to 4 experiments).

| Pₐₚₚ | EE27 | EE26 | Oftan Dexa | BSS Plus |
|---|---|---|---|---|
| (cm/sec) | (1 mg/ml DXM) | (1 mg/ml DXM) | (1 mg/ml DXM) | (0.1 mg/ml DXM) |
| Mean | 3.62 · 10⁻⁷ | 3.25 · 10⁻⁷ | 2.55 · 10⁻⁸ | 1.57 · 10⁻⁶ |
| SD | 1.60 · 10⁻⁷ | 8.70 · 10⁻⁸ | 1.29 · 10⁻⁸ | 9.68 · 10⁻⁷ |
| SEM | 7.99 · 10⁻⁸ | 4.35 · 10⁻⁸ | 6.44 · 10⁻⁹ | 6.84 · 10⁻⁷ |

1. An ophthalmic formulation in form of an oil-in-water microemulsion comprising oil particles dispersed in a continuous water phase, the formulation comprising
0.01-20.0 weight-%, preferably 0.1-2.0 weight-%, of a natural oil comprising triglycerides which comprise ≥50 weight-% of unsaturated fatty acid(s) with an aliphatic chain length of 16-20 carbon atoms, and
at least a first non-ionic emulsifier and a second non-ionic emulsifier, where both the first non-ionic emulsifier and the second non-ionic emulsifier comprise at least one structural unit with an aliphatic chain length of 16-20 carbon atoms originating from an unsaturated fatty acid,
wherein the formulation has a surface tension in a range of 40-43 mN/m at an emulsion-air interface at a temperature of 15-30° C.

2. The formulation according to claim 1, wherein the first non-ionic emulsifier and the second non-ionic emulsifier have a total molar concentration which is 80-150%, preferably 90-140%, more preferably 95-130%, of the total molar concentration of unsaturated fatty acid(s) of the natural oil triglycerides.

3. The formulation according to claim 1, wherein the oil particles have an average hydrodynamic diameter of 100 nm or less, preferably 50 nm or less, more preferably 30 nm or less.

4. The formulation according to claim 1, wherein the oil particles have a negative zeta potential, preferably from −100 mV to −0.5 mV, more preferably from −50 mV to −5 mV.

5. The formulation according to claim 1, wherein the formulation has an optical absorbance less than 0.3 absorbance units for a 1-cm path length in a wavelength range of 400-700 nm.

6. The formulation according to claim 1, wherein the natural oil is a fish oil or a plant-based oil, preferably a seed oil, more preferably a seed oil selected from canola oil, flaxseed (linseed) oil, raspberry seed oil, kiwi seed oil, candlenut tree (kukui) seed oil, borage seed oil, camelina sativa seed oil, sacha inchi seed oil, sea buckthorn seed oil, safflower seed oil, quinoa seed oil, watermelon seed oil, lime seed oil, orange seed oil, cucumber seed oil, pumpkin seed oil, strawberry seed oil, sunflower seed oil, walnut seed oil, boxthorn seed oil, evening primrose oil, noni seed oil, passion fruit seed oil, perilla seed oil, cherry seed oil, apple seed oil, black currant seed oil, currant seed oil, dog rose seed oil, cloudberry seed oil, wheat germ oil, blueberry seed oil, cranberry seed oil, bilberry seed oil, lingonberry seed oil, grape seed oil, or corn oil.

7. The formulation according to claim 1, wherein the unsaturated fatty acid comprises a monounsaturated fatty acid, such as palmitoleic acid (C16:1), oleic acid (C18:1), paullinic acid (C20:1) or any of their mixtures.

8. The formulation according to claim 1, wherein the unsaturated fatty acid comprises a polyunsaturated fatty acid, such as linoleic acid (C18:2), alpha-linolenic acid (C18:3), gamma-linolenic acid (C18:3), eicosapentaenoic acid (C20:5), or any of their mixtures.

9. The formulation according to claim 1, wherein the first non-ionic emulsifier is an oleyl ether or mono-oleate, preferably polyoxyethylene sorbitan mono-oleate, and/or the second non-ionic emulsifier is an oleyl ether or mono-oleate, preferably sorbitan mono-oleate.

10. The formulation according to claim 1, wherein the formulation is free of organic solvents and/or preservatives.

11. The formulation according to claim 1, wherein the formulation comprises one or more active agents.

12. The formulation according to claim 11, wherein the formulation comprises one or more hydrophobic active agents.

13. The formulation according to claim 11, wherein the one or more active agents are selected from anti-infective agents; corticosteroids; anti-inflammatory analgesics; glaucoma, mydriatic and cycloplegic drugs; antiallergic drugs; local anaesthetics; diagnostic agents; and/or immunomodulators.

14. The formulation according to claim 11, wherein the one or more active agents are selected from non-medicinal active agents.

15. (canceled)

16. A method to treat ocular surface disorder, the method comprising topically administering the formulation according to claim 1 to an eye manifesting symptom(s) of ocular surface disorder.

17. The method of claim 16, wherein the symptom(s) are manifested as tear film stability, impaired tear film retention and/or impaired tear film spreading, or tear film hyperosmolarity.

18. The method of claim 16, wherein the ocular surface disorder is selected from dry eye disease, blepharitis, Stevens-Johnson syndrome, Sjőgren's syndrome, ocular cicatricial pemphigoid or graft-versus-host disease.

19. The method of claim 16, wherein the formulation is administered topically in form of eye drops, eye gel, eye ointment, eye wash, eye mist, eyelid wipe or eye spray.
